(19) United States
(12) Patent Application Publication
(10) Pub. No.: US 2022/0088086 A1
(43) Pub. Date: Mar. 24, 2022

(54) Personalized Immunotherapies for Reduction of Brain Inflammation and Suicide Prevention

(71) Applicants: Thomas E. Ichim, Oceanside, CA (US); Timothy G. Dixon, Oceanside, CA (US); Famela Ramos, Oceanside, CA (US); Kalina O'Connor, Oceanside, CA (US)

(72) Inventors: Thomas E. Ichim, Oceanside, CA (US); Timothy G. Dixon, Oceanside, CA (US); Famela Ramos, Oceanside, CA (US); Kalina O'Connor, Oceanside, CA (US)

(73) Assignee: Therapeutic Solutions International, Inc., Oceanside, CA (US)

(21) Appl. No.: 17/030,416

(22) Filed: Sep. 24, 2020

(51) Int. Cl. A61K 35/51; A61K 35/16; A61K 35/19; A61K 38/24; A61K 38/095; A61K 31/09; A61K 38/19; A61K 38/18; A61K 9/51

(52) U.S. Cl. CPC A61K 35/51; A61K 35/16; A61K 35/19; A61K 38/24; A61K 38/191; A61K 31/09; A61K 38/193; A61K 38/1875; A61K 9/51; A61K 38/095

(57) ABSTRACT

Disclosed are means, methods and compositions of matter useful for reduction of brain inflammation and prevention of suicidal ideations and suicidal attempts. In one embodiment the invention provides utilization of autologous platelet rich plasma, alone, or admixed with regenerative/anti-inflammatory adjuvants, for reduction of neural inflammation. In one embodiment autologous PRP is admixed with oxytocin and administered intranasally in a patient at risk of suicidal ideation. In another embodiment, PRP is admixed with fortified and non-fortified nigella sativa oil and administered intranasally. Other embodiments include utilization of autologous stromal vascular fraction cells alone and/or admixed with regenerative/anti-inflammatory adjuvants.

### FIELD OF THE INVENTION

The invention pertains to the field of immunotherapy and more specifically the treatment of brain inflammation and the prevention of suicide.

### BACKGROUND

Suicide rates have been climbing in the United States in recent times. There is a need in the art for novel methods to treat this national emergency.

### SUMMARY

Embodiments herein are directed to methods of treating suicide ideations and attempts through the use of: assessing a biomarker associated with suicidal ideation or suicidal attempts; administering platelet rich plasma and/or cord blood plasma alone or in combination with an anti-inflammatory/regenerative adjuvant; d) assessing levels of said one or more biomarkers associated with said suicidal ideations/suicidal attempts; and e) adjusting dosage/frequency of administration of said platelet rich plasma alone or in combination with an anti- inflammatory/regenerative adjuvant.

### BRIEF DESCRIPTION OF THE DRAWINGS

FIG. 1 is a bar graph showing PRP and Pterostilbene reduce TNF-alpha levels.

FIG. 2 is a bar graph showing PRP and Pterostilbene reduce Interleukin-1 levels.

FIG. 3 is a bar graph showing PRP and Pterostilbene reduce Interleukin-6 levels.

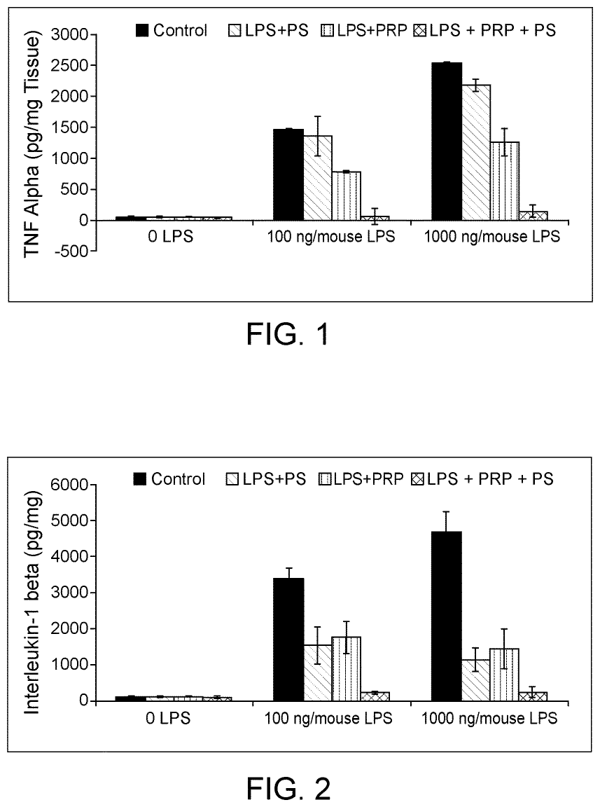

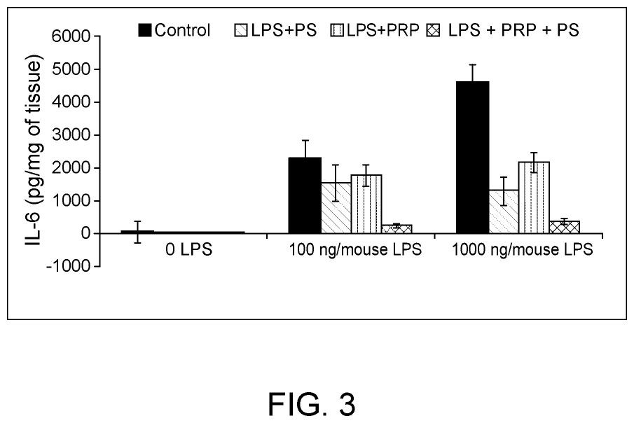

### DESCRIPTION OF THE INVENTION

As will be apparent to those of skill in the art upon reading this disclosure, each of the individual aspects described and illustrated herein has discrete components and features which may be readily separated from or combined with the features of any of the other several aspects without departing from the scope or spirit of the present disclosure. Any recited method can be carried out in the order of events recited or in any other order that is logically possible.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a neuropsychiatric disorder (including, but not limited to, PTSD, or a symptom thereof. Others are described elsewhere herein). The effect can be therapeutic in terms of a partial or complete cure of a disease, condition, symptom or adverse effect attributed to the disease, disorder, or condition. The term “treatment” as used herein covers any treatment of a neuropsychiatric disorder (including, but not limited to, PTSD or a symptom thereof), in a subject, particularly a human, and can include any one or more of the following:

(a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic (preventative) treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

Neuropsychiatric disorders are generally diseases, conditions, and disorders of affect, cognition, and/or behavior that can arise from an overt disorder in cerebral function or from indirect effects of extracerebral diseases and disorders. Neuropsychiatric disorders are a significant burden on society and can impair the health of those affected, as well as their ability to learn, work, and/or emotionally cope. They also can burden those not afflicted in that those affected often must rely on caregivers or other forms of assistance due to their inability to fully engage and function in normal work and life activities. Non-limiting examples of neuropsychiatric disorders include addiction, developmental conditions (e.g. attention deficit hyperactivity disorder (ADHD), autism, fetal alcohol syndrome, and tic disorders), eating disorders, degenerative disease (e.g. dementia, Parkinson's disease, and Alzheimer's disease), mood/affect disorders (e.g. bipolar disorder, depressions, and mania), neurotic disorders (e.g. obsessive compulsive disorder, trichotillomania, and anxiety disorders (including post-traumatic stress disorder (PTSD)), psychosis (e.g. schizophrenia), and sleep disorders (e.g. sleep apnea, narcolepsy, insomnia, and parasomnia).

In some embodiments of the invention, patients at risk for suicide and/or neuropsychiatric conditions are identified by on oxidative stress. Oxidative damage in the tissues and cells of an individual may be the result of reactive oxygen species (ROS), such as peroxides and oxygen radicals. Some level of ROS is normal in an organism and certain ROS species take part in normal biochemical pathways. However, excessive ROS levels cause oxidation of certain biomolecules in an individual, and the oxidation products, or derivatives therefrom, may appear in bodily fluids such as blood or urine. ROS can be produced from fungal or viral infection, ageing, UV radiation, pollution, excessive alcohol consumption, and cigarette smoking among other diseases. ROS can further cause age-related macular degeneration and cataracts. Of primary interest are the oxidation products of certain fatty acids and DNA, as the appearance of the oxidation products from fatty acids or DNA can be indicative of excessive ROS and the existence of oxidative damage at the cellular level. Oxidation of fatty acids in an organism is often referred to as “lipid peroxidation.” Further, ROS also includes the reactive nitrogen species (RNS), which includes nitric oxide radical NO and ONO.sub.2-. These reactive species cause “nitrative stress,” with RNS reaction products including such molecules as 3-nitrotyrosine. Since the RNS species are in effect ROS species, nitrative stress is normally lumped together with oxidative stress when referring to oxidative damage in individuals. In lipid peroxidation, it is the unsaturated fats that are most prone to oxidation, particularly arachidonic acid and linoleic acid with their polyunsaturated carbon chains. For example, oxidation of arachidonic acid and linoleic acid produces malondialdehyde (MDA) and 4-hydroxynonenal (4-HNE), amongst other products, which are secreted in the urine. MDA and 4-HNE can be measured in a urine sample as oxidative stress biomarkers. These biomarkers can be used to assess risk of suicide, as well as to predict response to therapies described in the current patent. An oxidative stress assay may comprise a specific malondialdehyde (MDA) or 4-hydroxyonenal (4HNE) method to quantify lipid peroxidation and/or a thiobarbituric acid reactive substances (TBARS) method to measure a broader range of substances oxidized to aldehydes and ketones due to the actions of free radicals. A ferrous reaction reagent suitable for use in assaying oxidative stress comprises 2-deoxyglucose, TBA, EDTA, and ferrous sulfate. Oxidized derivatives of amino acids in proteins are also biomarkers of oxidative stress. In principle, an oxidative stress biomarker can be any amino acid that has undergone oxidation or some other modification. For example, dityrosine and 3-nitrotyrosine are oxidative stress biomarkers produced by the reaction of tyrosine with peroxynitrite, or chloro-tyrosine, which is produced by the action of myeloperoxidase and is an inflammatory biomarker. Urinary 3-nitrotyrosine excretion is a urinary biomarker that reflects excessive ROS in an individual, such as ONO.sub.2-. 3-Nitrotyrosine is the major product of tyrosine oxidation, although it is not clear if tyrosine is oxidized when in free form or when part of a polypeptide. See, for example, Radi, R., “Nitric oxide, oxidants, and protein tyrosine nitration,” Proc. Natl, Acad. Sci., 101, 4003-4008 (2004). Further, oxidized sulfur- or selenium-containing amino acids (collectively referred to as “SSAA”) are oxidative stress biomarkers. Oxidized SSAA are amino acids in which the sulfur or selenium moiety has been oxidized to a higher oxidation state. Oxidized SSAA include, but are not limited to, cysteine, cystine, methionine, selenomethionine, selenocystine and selenocysteine in their various possible oxidation states. In general, high levels of any one of these biomarkers indicate that oxidative stress is occurring in an individual. Low levels of these biomarkers indicate a healthy individualAdditional oxidation and nitration products of lipids, proteins and DNA that find use as oxidative stress biomarkers include isoprostanes, 8-hydroxyguanosine and 8-hydroxy-2′deoxyguanosine. Oxidative damage to DNA can be evidenced by oxidation products of the most susceptible base, guanosine. The oxidation products that can be found at elevated levels in urine when excessive ROS are present include 8-hydroxyguanosine and 8-hydroxy-2′-guanosine. These substances have been shown to be useful biomarkers of oxidative stress. See, for example, Shigenaga, M. K., et al., “Urinary 8-hydroxy-2′deoxyguanosine as a biological marker of in vivo oxidative DNA damage,” Proc. Natl. Acad. Sci., 86, 9697-9701 (1989). Isoprostanes found in urine primarily consist of 8-iso-prostaglandin F.sub.2.alpha., referred to more simply herein as F2-isoprostane, or F2-isoP. F2-isoPs are chemically stable prostaglandin-like isomers, generated by the reaction of polyunsaturated fatty acids and ROS, and have been shown to be useful biomarkers for oxidative stress in an individual. See, for example, Cracowski, J.-L., et al., “Isoprostanes as a biomarker of lipid peroxidation in humans: physiology, pharmacology and clinical implications,” Trends Pharmacol. Sci., 23, 360-366 (2002)). Glutathione (GSH) is a tripeptide molecule that acts as an antioxidant, reducing various ROS species to become oxidized to the disulfide, GSSG. Since both the oxidized (GSH) and reduced (GSSG) species exist naturally, what is more important for health assessment is the ratio of GSH/GSSG. This ratio is about 30-100 in cytosol of cells, and about 3-10 in serum. The ratio decreases in the presence of oxidative stress. That is, there is an abnormally low level of GSH, and abnormally high level of GSSG, or both, causing the GSH/GSSG ratio to be lower than normal. See, in general, Frijhoff, J., et al., “Clinical relevance of biomarkers of oxidative stress,” Antioxid. Redox Signal., 23(14), 1144-70 (2015). Uric acid is a degradation product of purine, and is indicative of an inflammatory factor that increases oxidative stress and promotes activation of the renin angiotensin aldosterone system. Thus uric acid is a useful urinary biomarker indicative of oxidative stress and overall health. N-hexanoyl lysise (HEL), or more simply, hexanoyl-lysine adduct, is another lipid peroxidation biomarker. It is the product of omega-6 polyunsaturated fatty acid oxidation and is therefore elevated levels of HEL are indicative of excessive ROS in an individual. HEL concentration in human urine has been reported to be 22.9 nmol/L. See Sakai, K., et al., “Determination of HEL (hexanoyl-lysine adduct): a novel biomarker for omega-6 PUFA oxidation,” Subcell Biochem., 77, 61-72, (2014). In various embodiments, antioxidant capacity testing employs a CUPRAC (cupric reducing antioxidant capacity) method for measuring the sum of the antioxidant activity due to multiple species (uric acid, proteins, vitamins, dietary supplements) present in a urine sample (See e.g., Ozyurek, M., Guclu, K. and Apak, R., “The main and modified CUPRAC methods of antioxidant measurement,” Trends in Analytical Chemistry, 30: 652-664 (2011)). Alternatively, or additionally, modified methods can be used to specifically measure or to discriminate among uric acid, ascorbic proteins or other substances that contribute to the overall antioxidant capacity, thereby monitoring what is referred to as the “antioxidant reserve.” Several other biomarkers can be used to gauge antioxidant capacity and non-limiting examples are listed in TABLE 1 above. The CUPRAC method, and other methods that employ a redox indicator, directly measure the reaction of antioxidants with substances having the appropriate redox potential to effect a visible color change or a color interpretable by a simple colorimeter. A higher value for antioxidant power, that is, a greater level of biomarkers indicative of antioxidant capacity, indicates a healthy individual because the individual has compounds that can neutralize free radicals that cause oxidative damage and stress.

Certain embodiments commensurate in scope with the originally claimed invention are summarized below. These embodiments are not intended to limit the scope of the claimed invention, but rather these embodiments are intended only to provide a brief summary of possible forms of the invention. Indeed, the invention may encompass a variety of forms that may be similar to or different from the embodiments set forth below. One or more specific embodiments of the present subject matter will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The term “platelet plasma” as used herein refers to platelet rich plasma (PRP), human platelet lysate (HPL), and combinations thereof.

“Platelet rich plasma” (PRP) as described herein is a blood plasma that has been enriched with platelets. As a concentrated source of autologous platelets, PRP contains and releases several different growth factors and other cytokines that stimulate healing of bone and soft tissue. Components of PRP may include but are not limited to platelet-derived growth factor, transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, and combinations thereof.

PRP may be prepared by collection of the patient's whole blood (that is anticoagulated with citrate dextrose) before undergoing two stages of centrifugation designed to separate the PRP aliquot from platelet-poor plasma and red blood cells. In humans, a typical baseline blood platelet count may range from about 150,000 to about 450,000 platelets per .mu.l of blood, or about 200,000 platelets per .mu.l of blood. Therapeutic PRP may concentrate platelets in plasma by about five-fold. As such, PRP platelet count in PRP may range from about 750,000 to about 2.25.times.10.sup.6 platelets per .mu.l of PRP, or about 1.times.10.sup.6 platelets per .mu.l of blood. The PRP may then be used to prepare human platelet lysate.

Compositions of the present disclosure may comprise platelet plasma compositions from PRP, HPL, or combinations thereof, and either platelet plasma composition may be used to regenerate ovarian tissue for augmentation of fertility. Further, the platelet plasma composition may be used with or without concentrated bone marrow (BMAC). By way of example, when administered into ovarian tissue, about 0.05 to about 2.0 cc of platelet plasma composition may be used. Platelets are non-nucleated blood cells that as noted above are found in bone marrow and peripheral blood.

In various embodiments of the present invention, the platelet plasma composition may be obtained by sequestering platelets from whole blood or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires ovarian tissue regeneration. In some embodiments the lysate will act more rapidly than the PRP (or platelet poor plasma from bone marrow).

Human platelet lysate may be formed from but not limited to PRP, pooled platelets from humans, and cultured megakaryocytes from stem cell expansion technology. In some embodiments, HPL is from a commercial source. In some embodiments, HPL is prepared in the laboratory from platelet rich plasma (PRP), pooled platelets from humans, or cultured megakaryocytes from stem cell expansion technology.

Notably, platelet poor plasma that is derived from bone marrow has a greater platelet concentration than platelet rich plasma from blood, also known as platelet poor/rich plasma (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow.) In various embodiments, the platelet plasma composition, which may or may not be in the form of a lysate, may serve one or more of the following functions: (1) to release/provide growth factors and cytokines for tissue regeneration; (2) to reduce inflammation; (3) to attract/mobilize cell signaling; (4) to initiate repair of damaged/atrophied ovarian tissue through fibroblast growth factors (FGF); (5) to stabilize extracellular matrix in the ovary; (6) to stimulate maturation of immature oocytes; (7) to stimulate revascularization of fibrotic tissue; and (8) to stimulate oocyte receptivity to spermatozoa. Additionally, by combining platelet therapy with stem cells, there can be synergy with respect to augmentation of fertility.

In some embodiments in which the lysate is used, the cytokines may be concentrated in order to optimize their functional capacity. Concentration may be accomplished in two steps. First, blood may be obtained and concentrated to a volume that is 5-15% of what it was before concentration. Devices that may be used include but are not limited to a hemofilter or a hemo-concentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing 20-500 cc of peripheral blood, 40-250 cc of peripheral blood, or 60-100 cc of peripheral blood. The amount of blood that one should draw will depend on the extent of ovarian tissue degeneration.

In some embodiments, a method of generation of said PRP may be used according to U.S. Pat. No. 9,011,929, which is incorporated by reference herein in its entirety. In essence, a method may comprise separating PRP from whole blood by collecting whole blood from an animal or patient into a vacuum test tube containing sodium citrate, and primarily centrifuging the collected whole blood; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid; and collecting the PRP concentrated in a bottom layer by another blunt needle; mixing the PRP collected from the separating step with a calcium chloride solution by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at room temperature before mixing; and mixing the mixture of the PRP and the calcium chloride solution with the type I collagen, in an opaque phase, four times by another three-way connector.

In an exemplary embodiment of the disclosure, a method may comprise separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector.

The term “platelet-rich plasma” or “PRP” as used herein is a broad term which is used in its ordinary sense and is a concentration of platelets greater than the peripheral blood concentration suspended in a solution of plasma, or other excipient suitable for administration to a human or non-human animal including, but not limited to, isotonic sodium chloride solution, physiological saline, normal saline, dextrose 5% in water, dextrose 10% in water, Ringer solution, lactated Ringer solution, Ringer lactate, Ringer lactate solution, and the like. PRP compositions may be an autologous preparation from whole blood taken from the subject to be treated or, alternatively, PRP compositions may be prepared from a whole blood sample taken from a single donor source or from whole blood samples taken from multiple donor sources. In general, PRP compositions comprise platelets at a platelet concentration that is higher than the baseline concentration of the platelets in whole blood. In some embodiments, PRP compositions may further comprise WBCs at a WBC concentration that is higher than the baseline concentration of the WBCs in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration. In some embodiments, PRP compositions comprise elevated concentrations of platelets and WBCs and lower levels of RBCs and hemoglobin relative to their baseline concentrations. In some embodiments of PRP composition, only the concentration of platelets is elevated relative to the baseline concentration. Some embodiments of PRP composition comprise elevated levels of platelets and WBCs compared to baseline concentrations. In some embodiments, PRP compositions comprise elevated concentrations of platelets and lower levels of neutrophils relative to their baseline concentrations. Some embodiments of PRP composition comprise elevated levels of platelets and neutrophil-depleted WBCs compared to their baseline concentrations. In some embodiments of PRP, the ratio of lymphocytes and monocytes to neutrophils is significantly higher than the ratios of their baseline concentrations. The PRP formulation may include platelets at a level of between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood comprises at least 140,000 to 150,000 platelets and up to 400,000 to 500,000 platelets. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter. The WBC concentration is typically elevated in PRP compositions. For example, the WBC concentration may be between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000; about 10,000 and about 15,000; about 15,000 and about 20,000; about 20,000 and about 30,000; about 30,000 and about 50,000; about 50,000 and about 75,000; and about 75,000 and about 100,000. Among the WBCs in the PRP composition, the concentrations may vary by the cell type but, generally, each may be elevated. In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration. The concentrations of eosinophils in the PRP composition may be less than baseline, about 1.5 times baseline, about 2 times baseline, about 3 times baseline, about 5 times baseline, or higher.

In whole blood, the lymphocyte count is typically between 1,300 and 4,000 cells per microliter, but in other examples, the lymphocyte concentration may be between about 5,000 and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter.

In certain variations, the PRP composition may contain a specific concentration of neutrophils. The neutrophil concentration may vary between less than the baseline concentration of neutrophils to eight times than the baseline concentration of neutrophils. In some embodiments, the PRP composition may include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels are depleted to 1% or less than that found in whole blood. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, about 5,000 to about 20,000 per microliter, about 20,000 to about 40,000 per microliter, or about 40,000 to about 60,000 per microliter. In some embodiments, neutrophils are eliminated or substantially eliminated. Means to deplete blood products, such as PRP, of neutrophils is known and discussed in U.S. Pat. No. 7,462,268, which is incorporated herein by reference. Several embodiments are directed to PRP compositions in which levels of platelets and white blood cells are elevated compared to whole blood and in which the ratio of monocytes and/or lymphocytes to neutrophils is higher than in whole blood. The ratio of monocytes and/or lymphocytes to neutrophils may serve as an index to determine if a PRP formulation may be efficaciously used as a treatment for a particular disease or condition. PRP compositions where the ratio of monocytes and/or lymphocytes to neutrophils is increased may be generated by either lowering neutrophils levels, or by maintaining neutrophil levels while increasing levels of monocytes and/or lymphocytes. Several embodiments relate to a PRP formulation that contains 1.01 times, or higher, baseline platelets in combination with a 1.01 times, or higher, baseline white blood cells with the neutrophil component depleted at least 1% from baseline. In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women. In some embodiments, PRP compositions contain increased concentrations of growth factors and other cytokines. In several embodiments, PRP compositions may include increased concentrations of one or more of: platelet-derived growth factor, transforming growth factor beta, fibroblast growth factor, insulin-like growth factor, insulin-like growth factor 2, vascular endothelial growth factor, epidermal growth factor, interleukin-8, keratinocyte growth factor, and connective tissue growth factor. In some embodiments, the platelets collected in PRP are activated by thrombin and calcium chloride to induce the release of these growth factors from alpha granules. In some embodiments, a PRP composition is activated exogenously with thrombin and/or calcium to produce a gel that can be applied to an area to be treated. The process of exogenous activation, however, results in immediate release of growth factors. Other embodiments relate to activation of PRP via in vivo contact with collagen containing tissue at the treatment site. The in vivo activation of PRP results in slower growth factor release at the desired site.

In certain embodiments of the invention, the PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the tissue damage and/or the MI, the availability of prepared PRP, or any suitable combination of factors. Any suitable amount of blood may be collected. For example, about 1 cc to about 150 cc of blood or more may be drawn. More specifically, about 27 cc to about 110 cc or about 27 cc to about 55 cc of blood may be withdrawn. In some embodiments, the blood may be collected from a patient who may be presently suffering, or who has previously suffered from, connective tissue damage and/or an MI. PRP made from a patient's own blood may significantly reduce the risk of adverse reactions orinfection.

In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area. The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume.

In some examples, the blood may then be centrifuged using a gravitational platelet system, such as the Cell Factor Technologies GPS System.®. centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained.

As the PRP composition comprises activated platelets, active agents within the platelets are released. These agents include, but are not limited to, cytokines (e.g., IL-1B, IL-6, TNF-A), chemokines (e.g., ENA-78 (CXCL8), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-.beta. 1, 2, and 3, and VEGF).

Said PRP may be used to treat autologous regenerative cells prior to administration of said cells for stimulation of ovary regeneration and/or prevention of immunologically mediated abortions. One type of autologous regenerative cells are adipose stromal vascular fraction cells. Said stromal vascular fraction cells are obtained by the following steps; a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37.degree. C. shaking water bath for one hour then centrifuged for 5 min at 300 rcf; g) Following centrifugation, the stromal cells are resuspended within Isolyte in separate sterile 50 mL centrifuge tubes; h) The tubes are centrifuged for 5 min. at 300 rcf and the Isolyte is removed, leaving cell pellet; i) The pellets are resuspended in 40 ml of Isolyte, centrifuged again for 5 min at 300 rcf. The supernatant is again be removed; j) The cell pellets are combined and filtered through 100 .quadrature.m cell strainers into a sterile 50 ml centrifuge tube and centrifuged for 5 min at 300 rcf and the supernatant removed, leaving the pelleted adipose stromal cells. Means of combining PRP and SVF are known in the literature and incorporated by reference [3-7].

In some embodiments, the neutrophils are depleted by at least 5%, in some embodiments, the neutrophils are depleted by at least 10%, in some embodiments, the neutrophils are depleted by at least 15%, in some embodiments, the neutrophils are depleted by at least 20%, in some embodiments, the neutrophils are depleted by at least 25%, in some embodiments, the neutrophils are depleted by at least 30%, in some embodiments, the neutrophils are depleted by at least 35%, in some embodiments, the neutrophils are depleted by at least 40%, in some embodiments, the neutrophils are depleted by at least 45%, in some embodiments, the neutrophils are depleted by at least 50%, in some embodiments, the neutrophils are depleted by at least 55%, in some embodiments, the neutrophils are depleted by at least 60%, in some embodiments, the neutrophils are depleted by at least 65%, in some embodiments, the neutrophils are depleted by at least 70%, in some embodiments, the neutrophils are depleted by at least 75%, in some embodiments, the neutrophils are depleted by at least 80%, in some embodiments, the neutrophils are depleted by at least 85%, in some embodiments, the neutrophils are depleted by at least 90%, in some embodiments, the neutrophils are depleted by at least 95%, in some embodiments, the neutrophils are depleted by at least 95%. In some embodiments, the neutrophils in the platelet rich plasma are substantially removed.

### EXAMPLE 1: REDUCTION OF BRAIN TNF-ALPHA CONTENT BY PRP AND PTEROSTILBENE

BALB/c mice were anesthetized with isofluorane and administered pterstilbene (0.4 mg/mouse) and/or platelet rich plasma (5 microliter per mouse) via intranasal route subsequent to induction of systemic inflammation by intraperitoneal administration of endotoxin. Mice were sacrificed after 24 hours of treatment and quantification of cytokine (TNF-alpha) was performed by ELISA from brain homogenate tissue. As seen below, a synergistic reduction of TNF-alpha was observed.

### EXAMPLE 2: REDUCTION OF BRAIN IL-1 CONTENT BY PRP AND PTEROSTILBENE

BALB/c mice were anesthetized with isofluorane and administered pterstilbene (0.4 mg/mouse) and/or platelet rich plasma (5 microliter per mouse) via intranasal route subsequent to induction of systemic inflammation by intraperitoneal administration of endotoxin. Mice were sacrificed after 24 hours of treatment and quantification of cytokine (IL-1) was performed by ELISA from brain homogenate tissue. As seen below, a synergistic reduction of IL-1 was observed.

### EXAMPLE 3: REDUCTION OF BRAIN IL-6 CONTENT BY PRP AND PTEROSTILBENE

BALB/c mice were anesthetized with isofluorane and administered pterstilbene (0.4 mg/mouse) and/or platelet rich plasma (5 microliter per mouse) via intranasal route subsequent to induction of systemic inflammation by intraperitoneal administration of endotoxin. Mice were sacrificed after 24 hours of treatment and quantification of cytokine (IL-6) was performed by ELISA from brain homogenate tissue. As seen below, a synergistic reduction of IL-6 was observed.

A method of reducing suicidal ideations/suicidal attempts comprising the steps of: a) obtaining a patient with a propensity for suicidal ideation/suicidal attempts; b) assessing one or more markers associated with said suicidal ideations/suicidal attempts; c) administering to said patient platelet rich plasma and/or cord blood plasma alone or in combination with an anti-inflammatory/regenerative adjuvant; d) assessing levels of said one or more markers associated with said suicidal ideations/suicidal attempts; and e) adjusting dosage/frequency of administration of said platelet rich plasma alone or in combination with an anti-inflammatory/regenerative adjuvant.

Further embodiments are directed to methods wherein propensity for suicidal ideation/suicidal attempts is associated with augmentation of inflammatory cytokines and/or mediators in a biological fluid as compared to an age- matched control.

Further embodiments are directed to methods wherein said biological fluid is blood, plasma, or serum.

Further embodiments are directed to methods wherein said biological fluid is urine.

Further embodiments are directed to methods wherein said biological fluid is saliva.

Further embodiments are directed to methods wherein said biological fluid is tears.

Further embodiments are directed to methods wherein said biological fluid is bronchiolar lay age fluid.

Further embodiments are directed to methods wherein said biological fluid is cerebral spinal fluid.

Further embodiments are directed to methods wherein said inflammatory cytokine is IL-1 beta.

Further embodiments are directed to methods wherein said inflammatory cytokine is IL-6.

Further embodiments are directed to methods wherein said inflammatory cytokine is IL-7.

Further embodiments are directed to methods wherein said inflammatory cytokine is IL-8.

Further embodiments are directed to methods wherein said inflammatory cytokine is IL-12.

Further embodiments are directed to methods wherein said inflammatory cytokine is IL-15.

Further embodiments are directed to methods wherein said inflammatory cytokine is IL-17.

Further embodiments are directed to methods wherein said inflammatory cytokine is IL-18.

Further embodiments are directed to methods wherein said inflammatory cytokine is IL-21.

Further embodiments are directed to methods wherein said inflammatory cytokine is IL-9.

Further embodiments are directed to methods wherein said inflammatory cytokine is IL-27.

Further embodiments are directed to methods wherein said inflammatory cytokine is IL-23.

Further embodiments are directed to methods wherein said inflammatory cytokine is IL-33.

Further embodiments are directed to methods wherein said inflammatory cytokine is BAFF.

Further embodiments are directed to methods wherein said inflammatory cytokine is 4-1 BBL.

Further embodiments are directed to methods wherein said inflammatory cytokine is TNFSF8.

Further embodiments are directed to methods wherein said inflammatory cytokine is CD40LG.

Further embodiments are directed to methods wherein said inflammatory cytokine is CD70.

Further embodiments are directed to methods wherein said inflammatory cytokine is CD95L.

Further embodiments are directed to methods wherein said inflammatory cytokine is TNFSF8.

Further embodiments are directed to methods wherein said inflammatory cytokine is EDA-A1.

Further embodiments are directed to methods wherein said inflammatory cytokine is TNFSF15.

Further embodiments are directed to methods wherein said inflammatory cytokine is TNF-beta.

Further embodiments are directed to methods wherein said inflammatory cytokine is LTB.

Further embodiments are directed to methods wherein said inflammatory cytokine is TNF-alpha.

Further embodiments are directed to methods wherein said inflammatory cytokine is TNFSF10.

Further embodiments are directed to methods wherein said inflammatory cytokine is TNFSF11.

Further embodiments are directed to methods wherein said inflammatory cytokine is TNFSF12.

Further embodiments are directed to methods wherein said inflammatory cytokine is TNFSF13.

Further embodiments are directed to methods wherein said inflammatory cytokine is TNFSF4.

Further embodiments are directed to methods wherein said inflammatory cytokine is TNFSF10.

Further embodiments are directed to methods wherein said inflammatory cytokine is IFNA1.

Further embodiments are directed to methods wherein said inflammatory cytokine is IFNA10.

Further embodiments are directed to methods wherein said inflammatory cytokine is IFNA13.

Further embodiments are directed to methods wherein said inflammatory cytokine is IFNA14.

Further embodiments are directed to methods wherein said inflammatory cytokine is IFNA2.

Further embodiments are directed to methods wherein said inflammatory cytokine is IFNA4.

Further embodiments are directed to methods wherein said inflammatory cytokine is IFNA7.

Further embodiments are directed to methods wherein said inflammatory cytokine is IFNB1.

Further embodiments are directed to methods wherein said inflammatory cytokine is IFNE.

Further embodiments are directed to methods wherein said inflammatory cytokine is IFNG.

Further embodiments are directed to methods wherein said inflammatory cytokine is IFNZ.

Further embodiments are directed to methods wherein said inflammatory cytokine is IFNA8.

Further embodiments are directed to methods wherein said inflammatory cytokine is IFNA5.

Further embodiments are directed to methods wherein said inflammatory cytokine is IFNW1.

Further embodiments are directed to methods wherein said inflammatory cytokine is CLCF1.

Further embodiments are directed to methods wherein said inflammatory cytokine is CNTF.

Further embodiments are directed to methods wherein said inflammatory cytokine is IFNW1.

Further embodiments are directed to methods wherein said inflammatory cytokine is IL-11.

Further embodiments are directed to methods wherein said inflammatory cytokine is IL-31.

Further embodiments are directed to methods wherein said inflammatory cytokine is leptin.

Further embodiments are directed to methods wherein said inflammatory cytokine is LIF.

Further embodiments are directed to methods wherein said inflammatory cytokine is OSM.

Further embodiments are directed to methods wherein said inflammatory cytokine is IL-1A.

Further embodiments are directed to methods wherein said inflammatory cytokine is IL-1B.

Further embodiments are directed to methods wherein said inflammatory cytokine is IL1F10.

Further embodiments are directed to methods wherein said inflammatory cytokine is IL1F3.

Further embodiments are directed to methods wherein said inflammatory cytokine is IL1F5.

Further embodiments are directed to methods wherein said inflammatory cytokine is IL-1A.

Further embodiments are directed to methods wherein said inflammatory cytokine is ILF6.

Further embodiments are directed to methods wherein said inflammatory cytokine is IL1F8.

Further embodiments are directed to methods wherein said inflammatory cytokine is IL1RL2.

Further embodiments are directed to methods wherein said inflammatory cytokine is IL1F9.

Further embodiments are directed to methods wherein said inflammatory mediator is a chemokine.

Further embodiments are directed to methods wherein said chemokine is CCL1.

Further embodiments are directed to methods wherein said chemokine is CCL11.

Further embodiments are directed to methods wherein said chemokine is CCL12.

Further embodiments are directed to methods wherein said chemokine is MCP4.

Further embodiments are directed to methods wherein said chemokine is CCL14.

Further embodiments are directed to methods wherein said chemokine is CCL15.

Further embodiments are directed to methods wherein said chemokine is CCL16.

Further embodiments are directed to methods wherein said chemokine is TARC.

Further embodiments are directed to methods wherein said chemokine is CCL18.

Further embodiments are directed to methods wherein said chemokine is CCL19.

Further embodiments are directed to methods wherein said chemokine is MCP1.

Further embodiments are directed to methods wherein said chemokine is CCL20.

Further embodiments are directed to methods wherein said chemokine is CCL21.

Further embodiments are directed to methods wherein said chemokine is MDC.

Further embodiments are directed to methods wherein said chemokine is CCL23.

Further embodiments are directed to methods wherein said chemokine is CCL24.

Further embodiments are directed to methods wherein said chemokine is CCL25.

Further embodiments are directed to methods wherein said chemokine is CCL26.

Further embodiments are directed to methods wherein said chemokine is CCL27.

Further embodiments are directed to methods wherein said chemokine is CCL28.

Further embodiments are directed to methods wherein said chemokine is CCL3.

Further embodiments are directed to methods wherein said chemokine is CCL3L3.

Further embodiments are directed to methods wherein said chemokine is CCL4.

Further embodiments are directed to methods wherein said chemokine is CCL1.

Further embodiments are directed to methods wherein said chemokine is LAG-1.

Further embodiments are directed to methods wherein said chemokine is CCL5.

Further embodiments are directed to methods wherein said chemokine is CCL6.

Further embodiments are directed to methods wherein said chemokine is CCL7.

Further embodiments are directed to methods wherein said chemokine is CCL8.

Further embodiments are directed to methods wherein said chemokine is CCL9.

Further embodiments are directed to methods wherein said chemokine is CX3CL1.

Further embodiments are directed to methods wherein said chemokine is CXCL1.

Further embodiments are directed to methods wherein said chemokine is CXCL10.

Further embodiments are directed to methods wherein said chemokine is CXCL11.

Further embodiments are directed to methods wherein said chemokine is CXCL12.

Further embodiments are directed to methods wherein said chemokine is CXCL13.

Further embodiments are directed to methods wherein said chemokine is CXCL14.

Further embodiments are directed to methods wherein said chemokine is CXCL15.

Further embodiments are directed to methods wherein said chemokine is CXCL16.

Further embodiments are directed to methods wherein said chemokine is CXCL17.

Further embodiments are directed to methods wherein said chemokine is MIP-2.

Further embodiments are directed to methods wherein said chemokine is CXCL3.

Further embodiments are directed to methods wherein said chemokine is CXCL4.

Further embodiments are directed to methods wherein said chemokine is CXCL5.

Further embodiments are directed to methods wherein said chemokine is CXCL6.

Further embodiments are directed to methods wherein said chemokine is Ppbp.

Further embodiments are directed to methods wherein said chemokine is CXCL9.

Further embodiments are directed to methods wherein said chemokine is XCL1.

Further embodiments are directed to methods wherein said chemokine is XCL2.

Further embodiments are directed to methods wherein said chemokine is FAM19A1.

Further embodiments are directed to methods wherein said chemokine is FAM19A2.

Further embodiments are directed to methods wherein said chemokine is FAM19A3.

Further embodiments are directed to methods wherein said chemokine is FAM19A4.

Further embodiments are directed to methods wherein said chemokine is FAM19A5.

Further embodiments are directed to methods wherein said inflammatory mediator is kynurenin.

Further embodiments are directed to methods wherein said inflammatory mediator is spermidine/spermine N1-acetyltransferase 1 (SAT1).

Further embodiments are directed to methods wherein said inflammatory mediator is forkhead box N3 (FOXN3).

Further embodiments are directed to methods wherein said inflammatory mediator is guanylate binding protein 1 (GBP1).

Further embodiments are directed to methods wherein said inflammatory mediator is phosphoinositide-3-kinase regulatory subunit 5 (PIK3R5).

Further embodiments are directed to methods wherein said inflammatory mediator is apolipoprotein L2 (APOL2).

Further embodiments are directed to methods wherein said inflammatory mediator is ATPase H+ transporting lysosomal 9 kDa, V0 subunit e1 (ATP6V0E1).

Further embodiments are directed to methods wherein said inflammatory mediator is GRINL1A complex locus (GCOM1).

Further embodiments are directed to methods wherein said inflammatory mediator is lipoma HMGIC fusion partner (LHFP).

Further embodiments are directed to methods wherein said inflammatory mediator is lipase A (LIPA).

Further embodiments are directed to methods wherein said inflammatory mediator is myristoylated alanine-rich protein kinase C substrate (MARCKS).

Further embodiments are directed to methods wherein said inflammatory mediator is 6-phosphogluconolactonase (PGLS).

Further embodiments are directed to methods wherein said inflammatory mediator is phosphatase and tensin homolog (PTEN).

Further embodiments are directed to methods wherein said inflammatory mediator is reversion-inducing-cysteine-rich protein with kazal motifs (RECK).

Further embodiments are directed to methods wherein said inflammatory mediator is ABCB1.

Further embodiments are directed to methods wherein said inflammatory mediator is ACACB.

Further embodiments are directed to methods wherein said inflammatory mediator is ACAT1.

Further embodiments are directed to methods wherein said inflammatory mediator is ACHE.

Further embodiments are directed to methods wherein said inflammatory mediator is ADRB1.

Further embodiments are directed to methods wherein said inflammatory mediator is ADRB2.

Further embodiments are directed to methods wherein said inflammatory mediator is AKT1.

Further embodiments are directed to methods wherein said inflammatory mediator is AKT2.

Further embodiments are directed to methods wherein said inflammatory mediator is ANGPT1.

Further embodiments are directed to methods wherein said inflammatory mediator is APOB.

Further embodiments are directed to methods wherein said inflammatory mediator is APOH.

Further embodiments are directed to methods wherein said inflammatory mediator is APOL3.

Further embodiments are directed to methods wherein said inflammatory mediator is APOL4

Further embodiments are directed to methods wherein said inflammatory mediator is AVEN.

Further embodiments are directed to methods wherein said inflammatory mediator is CETP.

Further embodiments are directed to methods wherein said inflammatory mediator is CHAT.

Further embodiments are directed to methods wherein said inflammatory mediator is CHKB.

Further embodiments are directed to methods wherein said inflammatory mediator is CPT1A.

Further embodiments are directed to methods wherein said inflammatory mediator is CRHR2.

Further embodiments are directed to methods wherein said inflammatory mediator is DBH.

Further embodiments are directed to methods wherein said inflammatory mediator is DRD3.

Further embodiments are directed to methods wherein said inflammatory mediator is DRD4.

Further embodiments are directed to methods wherein said inflammatory mediator is DRD5.

Further embodiments are directed to methods wherein said inflammatory mediator is DTNBP1.

Further embodiments are directed to methods wherein said inflammatory mediator is FLJ32252.

Further embodiments are directed to methods wherein said inflammatory mediator is FLT1.

Further embodiments are directed to methods wherein said inflammatory mediator is GABRA2.

Further embodiments are directed to methods wherein said inflammatory mediator is GAL.

Further embodiments are directed to methods wherein said inflammatory mediator is GNAO1.

Further embodiments are directed to methods wherein said inflammatory mediator is GYS2.

Further embodiments are directed to methods wherein said inflammatory mediator is ABCA1.

Further embodiments are directed to methods wherein said inflammatory mediator is FLJ10357.

Further embodiments are directed to methods wherein said inflammatory mediator is CASC1.

Further embodiments are directed to methods wherein said inflammatory mediator is DHR9.

Further embodiments are directed to methods wherein said inflammatory mediator is DISC1.

Further embodiments are directed to methods wherein said inflammatory mediator is EIF2AK2.

Further embodiments are directed to methods wherein said inflammatory mediator is MAP3K3.

Further embodiments are directed to methods wherein said inflammatory mediator is MT-ND6.

Further embodiments are directed to methods wherein said inflammatory mediator is RBM-47.

Further embodiments are directed to methods wherein said inflammatory mediator is RPTOR.

Further embodiments are directed to methods wherein said inflammatory mediator is RICTOR.

Further embodiments are directed to methods wherein said inflammatory mediator is SAMD9L.

Further embodiments are directed to methods wherein said inflammatory mediator is SCARF-1.

Further embodiments are directed to methods wherein said inflammatory mediator is SLC36A1.

Further embodiments are directed to methods wherein said inflammatory mediator is STAT1.

Further embodiments are directed to methods wherein said inflammatory mediator is COX5B.

Further embodiments are directed to methods wherein said inflammatory mediator is SMARCA1.

Further embodiments are directed to methods wherein said inflammatory mediator is UBA6.

Further embodiments are directed to methods wherein said inflammatory mediator is ZC3HAV1.

Further embodiments are directed to methods wherein said inflammatory mediator is TNK2.

Further embodiments are directed to methods wherein said inflammatory mediator is CD24.

Further embodiments are directed to methods wherein said inflammatory mediator is ATP13A2.

Further embodiments are directed to methods wherein said inflammatory mediator is EPHX1.

Further embodiments are directed to methods wherein said inflammatory mediator is HTRA1.

Further embodiments are directed to methods wherein said inflammatory mediator is LEPR.

Further embodiments are directed to methods wherein said inflammatory mediator is SPTBN1.

Further embodiments are directed to methods wherein said inflammatory mediator is MBNL2.

Further embodiments are directed to methods wherein said inflammatory mediator is OR2J3.

Further embodiments are directed to methods wherein said inflammatory mediator is RHEB.

Further embodiments are directed to methods wherein said inflammatory mediator is GRINA.

Further embodiments are directed to methods wherein said inflammatory mediator is KCNJ2.

Further embodiments are directed to methods wherein said inflammatory mediator is TOP1.

Further embodiments are directed to methods wherein said inflammatory mediator is GYS2.

Further embodiments are directed to methods wherein said inflammatory mediator is ICAM1.

Further embodiments are directed to methods wherein said inflammatory mediator is INSR.

Further embodiments are directed to methods wherein said inflammatory mediator is KDR.

Further embodiments are directed to methods wherein said inflammatory mediator is LDLR.

Further embodiments are directed to methods wherein said inflammatory mediator is LIPE.

Further embodiments are directed to methods wherein said inflammatory mediator is LIPF.

Further embodiments are directed to methods wherein said inflammatory mediator is LOC391530.

Further embodiments are directed to methods wherein said inflammatory mediator is OLR1.

Further embodiments are directed to methods wherein said inflammatory mediator is OXT.

Further embodiments are directed to methods wherein said inflammatory mediator is PIK3C2G.

Further embodiments are directed to methods wherein said inflammatory mediator is PIK3C3.

Further embodiments are directed to methods wherein said inflammatory mediator is PIK3R1.

Further embodiments are directed to methods wherein said inflammatory mediator is PPARG.

Further embodiments are directed to methods wherein said inflammatory mediator is PRKAA1.

Further embodiments are directed to methods wherein said inflammatory mediator is PRKAB1.

Further embodiments are directed to methods wherein said inflammatory mediator is RARB.

Further embodiments are directed to methods wherein said inflammatory mediator is RARG.

Further embodiments are directed to methods wherein said inflammatory mediator is RXRA.

Further embodiments are directed to methods wherein said inflammatory mediator is SCARB2.

Further embodiments are directed to methods wherein said inflammatory mediator is SELE.

Further embodiments are directed to methods wherein said inflammatory mediator is SSTR3.

Further embodiments are directed to methods wherein said inflammatory mediator is leukotriene B4.

Further embodiments are directed to methods wherein said inflammatory mediator is Decanoylcarnitine.

Further embodiments are directed to methods wherein said inflammatory mediator is Butenylcarnitine.

Further embodiments are directed to methods wherein said inflammatory mediator is hydroxybutyrylcarnitine.

Further embodiments are directed to methods wherein said inflammatory mediator is 2-aminooctanoate.

Further embodiments are directed to methods wherein said inflammatory mediator is hexadecanedioate.

Further embodiments are directed to methods wherein said inflammatory mediator is 3-hydroxybutyrate.

Further embodiments are directed to methods wherein said inflammatory mediator is stearate (18:0).

Further embodiments are directed to methods wherein said inflammatory mediator is oleate(18:1n9).

Further embodiments are directed to methods wherein said inflammatory mediator is 10-heptadecenoate (17:1n7).

Further embodiments are directed to methods wherein said inflammatory mediator is 10-nonadecenoate (19:1n9).

Further embodiments are directed to methods wherein said inflammatory mediator is margarate (17:0).

Further embodiments are directed to methods wherein said inflammatory mediator is palmitate (16:0).

Further embodiments are directed to methods wherein said inflammatory mediator is arachidate (20:0).

Further embodiments are directed to methods wherein said inflammatory mediator is lysoPC a C26:0.

Further embodiments are directed to methods wherein said inflammatory mediator is lysoPC a C18:2.

Further embodiments are directed to methods wherein said inflammatory mediator is lysoPC a C18:1.

Further embodiments are directed to methods wherein said inflammatory mediator is 2-linoleoylglycerophosphoethanolamine.

Further embodiments are directed to methods wherein said inflammatory mediator is 2-1-oleoylglycerophosphoinositol.

Further embodiments are directed to methods wherein said inflammatory mediator is 2-linoleoylglycerophosphocholine.

Further embodiments are directed to methods wherein said inflammatory mediator is 1-palmitoylglycerophosphoinositol.

Further embodiments are directed to methods wherein said inflammatory mediator is 1-linoleoylglycerophosphoethanolamine.

Further embodiments are directed to methods wherein said inflammatory mediator is 2-palmitoleoylglycerophosphocholine.

Further embodiments are directed to methods wherein said inflammatory mediator is 1-arachidonoylglycerophosphoethanolamine.

Further embodiments are directed to methods wherein said inflammatory mediator is 1-linolenoylglycerophosphocholine (18:3n3).

Further embodiments are directed to methods wherein said inflammatory mediator is 1-docosapentaenoylglycerophosphocholine (22:5n3).

Further embodiments are directed to methods wherein said inflammatory mediator is 5-dodecenoate (12:1n7).

Further embodiments are directed to methods wherein said inflammatory mediator is glycerophosphorylcholine (GPC).

Further embodiments are directed to methods wherein said inflammatory mediator is dihomo-linoleate (20:2n6).

Further embodiments are directed to methods wherein said inflammatory mediator is docosadienoate (22:2n6).

Further embodiments are directed to methods wherein said inflammatory mediator is taurocholenate sulfate.

Further embodiments are directed to methods wherein said inflammatory mediator is dihomo-linoleate (20:2n6).

Further embodiments are directed to methods wherein said inflammatory mediator is sphingosine.

Further embodiments are directed to methods wherein said inflammatory mediator is SMC18:0.

Further embodiments are directed to methods wherein said inflammatory mediator is SMC24:1.

Further embodiments are directed to methods wherein said inflammatory mediator is SM(OH)C14:1.

Further embodiments are directed to methods wherein said inflammatory mediator is SM C24:0.

Further embodiments are directed to methods wherein said inflammatory mediator is cortisol.

Further embodiments are directed to methods wherein said inflammatory mediator is creatine.

Further embodiments are directed to methods wherein said inflammatory mediator is glutamine.

Further embodiments are directed to methods wherein said inflammatory mediator is glutamate.

Further embodiments are directed to methods wherein said inflammatory mediator is serine.

Further embodiments are directed to methods wherein said inflammatory mediator is Glycine.

Further embodiments are directed to methods wherein said inflammatory mediator is betaine.

Further embodiments are directed to methods wherein said inflammatory mediator is 3-hydroxyisobutyrate.

Further embodiments are directed to methods wherein said inflammatory mediator is valine.

Further embodiments are directed to methods wherein said inflammatory mediator is 3-methyl-2-oxobutyrate.

Further embodiments are directed to methods wherein said inflammatory mediator is alpha-hydroxyisovalerate.

Further embodiments are directed to methods wherein said inflammatory mediator is 3-methylglutarylcarnitine (C6).

Further embodiments are directed to methods wherein said inflammatory mediator is alpha-Aminoadipic acid.

Further embodiments are directed to methods wherein said inflammatory mediator is pipecolate.

Further embodiments are directed to methods wherein said inflammatory mediator is methionine.

Further embodiments are directed to methods wherein said inflammatory mediator is 2-aminobutyrate.

Further embodiments are directed to methods wherein said inflammatory mediator is 3-(4-hydroxyphenyl)lactate

Further embodiments are directed to methods wherein said inflammatory mediator is indolepropionate.

Further embodiments are directed to methods wherein said inflammatory mediator is C-glycosyltryptophan.

Further embodiments are directed to methods wherein said inflammatory mediator is citrulline.

Further embodiments are directed to methods wherein said inflammatory mediator is ornithine.

Further embodiments are directed to methods wherein said inflammatory mediator is bilirubin.

Further embodiments are directed to methods wherein said inflammatory mediator is biliverdin.

Further embodiments are directed to methods wherein said inflammatory mediator is N1-Methyl-2-pyridone-5-carboxamide.

Further embodiments are directed to methods wherein said inflammatory mediator is hypoxanthine.

Further embodiments are directed to methods wherein said inflammatory mediator is adenosine 5′-monophosphate (AMP).

Further embodiments are directed to methods wherein said inflammatory mediator is N2,N2-dimethylguanosine.

Further embodiments are directed to methods wherein said inflammatory mediator is uridine.

Further embodiments are directed to methods wherein said inflammatory mediator is pseudouridine.

Further embodiments are directed to methods wherein said inflammatory mediator is 5-methyluridine(ribothymidine).

Further embodiments are directed to methods wherein said inflammatory mediator is gamma-glutamyltyrosine.

Further embodiments are directed to methods wherein said inflammatory mediator is gamma-glutamylmethionine.

Further embodiments are directed to methods wherein said inflammatory mediator is bradykinin.

Further embodiments are directed to methods wherein said inflammatory mediator is HWESASXX.

Further embodiments are directed to methods wherein said inflammatory mediator is bradykinin, des-arg(9).

Further embodiments are directed to methods wherein said inflammatory mediator is bradykinin, hydroxy-pro(3).

Further embodiments are directed to methods wherein said inflammatory mediator is CRP.

Further embodiments are directed to methods wherein said inflammatory mediator is lactate.

Further embodiments are directed to methods wherein said inflammatory mediator is pyruvate.

Further embodiments are directed to methods wherein said inflammatory mediator is hypoxanthine.

Further embodiments are directed to methods wherein said inflammatory mediator is 1-methyl-2-piperidinecarboxylic acid.

Further embodiments are directed to methods wherein propensity for suicidal ideation is quantified using a psychological scale.

Further embodiments are directed to methods wherein said psychological scale is a standard scale currently utilized in routine psychological practice.

Further embodiments are directed to methods wherein said scale is the SAD PERSONS Scale.

Further embodiments are directed to methods wherein said scale is the Manchester Self-Harm Rule (MSHR).

Further embodiments are directed to methods wherein said scale is the ReACT scale.

Further embodiments are directed to methods wherein said scale is the Sodersjukhuset Self Harm Rule.

Further embodiments are directed to methods wherein said scale is the Beck Hopelessness Scale.

Further embodiments are directed to methods wherein propensity for suicidal ideation is quantified by assessment of numbers of circulating endothelial progenitor cells in the blood, wherein a decreased number of endothelial progenitor cells in a patient, compared to an age matched control is indicative of suicidal risk.

Further embodiments are directed to methods wherein said endothelial progenitor cells are capable of forming endothelial colonies when plated in vitro.

Further embodiments are directed to methods wherein said endothelial progenitor cells are capable of forming endothelial tubes when plated in a semisolid media.

Further embodiments are directed to methods wherein said endothelial progenitor cells express CD34.

Further embodiments are directed to methods wherein said endothelial progenitor cells express CD133.

Further embodiments are directed to methods wherein said endothelial progenitor cells express stem cell factor receptor.

Further embodiments are directed to methods wherein said endothelial progenitor cells express VEGF-receptor.

Further embodiments are directed to methods wherein said endothelial progenitor cells express c-met.

Further embodiments are directed to methods wherein said endothelial progenitor cells express IL-3 receptor.

Further embodiments are directed to methods wherein said endothelial progenitor cells express TNF-alpha receptor p55.

Further embodiments are directed to methods wherein said endothelial progenitor cells express TNF-alpha receptor p75.

Further embodiments are directed to methods wherein said endothelial progenitor cells express nerve growth factor receptor.

Further embodiments are directed to methods wherein said endothelial progenitor cells express TNF-alpha receptor p55.

Further embodiments are directed to methods wherein said endothelial progenitor cells express VE-Cadherin.

Further embodiments are directed to methods wherein said endothelial progenitor cells express CD31.

Further embodiments are directed to methods wherein said endothelial progenitor cells express CD117.

Further embodiments are directed to methods wherein said endothelial progenitor cells express CXCR4.

Further embodiments are directed to methods wherein said endothelial progenitor cells express CD146.

Further embodiments are directed to methods wherein said endothelial progenitor cells express PLVAP.

Further embodiments are directed to methods wherein said endothelial progenitor cells express S1P1/EDG-1.

Further embodiments are directed to methods wherein said endothelial progenitor cells express S1P2/EDG-5.

Further embodiments are directed to methods wherein said endothelial progenitor cells express S1P3/EDG-3.

Further embodiments are directed to methods wherein said endothelial progenitor cells express S1P4/EDG-6.

Further embodiments are directed to methods wherein said endothelial progenitor cells express S1P5/EDG-8.

Further embodiments are directed to methods wherein said endothelial progenitor cells express E-Selectin/CD62E.

Further embodiments are directed to methods wherein said endothelial progenitor cells express E-Selectin/CD62P.

Further embodiments are directed to methods wherein said endothelial progenitor cells express Tie-2.

Further embodiments are directed to methods wherein said endothelial progenitor cells express VCAM-1/CD106.

Further embodiments are directed to methods wherein propensity towards suicide is associated with increased number of inflammatory T cells.

Further embodiments are directed to methods wherein said inflammatory T cells are T cells capable of activating macrophages.

Further embodiments are directed to methods wherein said T cells capable of activating said macrophages secrete interferon gamma, wherein said interferon gamma is involved in activation of said macrophages.

Further embodiments are directed to methods wherein said T cells capable of activating said macrophages secrete interleukin-1, wherein said interleukin-1 is involved in activation of said macrophages.

Further embodiments are directed to methods wherein said T cells capable of activating said macrophages secrete TNF-alpha, wherein said TNF-alpha is involved in activation of said macrophages.

Further embodiments are directed to methods wherein said T cells capable of activating said macrophages secrete interleukin-6, wherein said interleukin-6 is involved in activation of said macrophages.

Further embodiments are directed to methods wherein said T cells capable of activating said macrophages secrete interleukin-8, wherein said interleukin-8 is involved in activation of said macrophages.

Further embodiments are directed to methods wherein said T cells capable of activating said macrophages secrete interleukin-9, wherein said interleukin-9 is involved in activation of said macrophages.

Further embodiments are directed to methods wherein said T cells capable of activating said macrophages secrete interleukin-11, wherein said interleukin-11 is involved in activation of said macrophages.

Further embodiments are directed to methods wherein said T cells capable of activating said macrophages secrete interleukin-12, wherein said interleukin-12 is involved in activation of said macrophages.

Further embodiments are directed to methods wherein said T cells capable of activating said macrophages secrete interleukin-15, wherein said interleukin-15 is involved in activation of said macrophages.

Further embodiments are directed to methods wherein said T cells capable of activating said macrophages secrete interleukin-18, wherein said interleukin-18 is involved in activation of said macrophages.

Further embodiments are directed to methods wherein said T cells capable of activating said macrophages secrete interleukin-17, wherein said interleukin-17 is involved in activation of said macrophages.

Further embodiments are directed to methods wherein said T cells capable of activating said macrophages secrete interleukin-21, wherein said interleukin-21 is involved in activation of said macrophages.

Further embodiments are directed to methods wherein said T cells capable of activating said macrophages secrete interleukin-22, wherein said interleukin-22 is involved in activation of said macrophages.

Further embodiments are directed to methods wherein said T cells capable of activating said macrophages secrete interleukin-23, wherein said interleukin-23 is involved in activation of said macrophages.

Further embodiments are directed to methods wherein said T cells capable of activating said macrophages secrete interleukin-27, wherein said interleukin-27 is involved in activation of said macrophages.

Further embodiments are directed to methods wherein said T cells capable of activating said macrophages secrete interleukin-33, wherein said interleukin-33 is involved in activation of said macrophages.

Further embodiments are directed to methods wherein said T cells capable of activating said macrophages secrete VIP-1, wherein said VIP-1 is involved in activation of said macrophages.

Further embodiments are directed to methods wherein said T cells capable of activating said macrophages secrete PACAP, wherein said PACAP is involved in activation of said macrophages.

Further embodiments are directed to methods wherein said inflammatory T cells are Th17 cells.

Further embodiments are directed to methods wherein said Th17 cells express the transcription factor RoR gamma.

Further embodiments are directed to methods wherein said Th17 cells express angiogenic markers.

Further embodiments are directed to methods wherein said angiogenic marker is Plexin D1.

Further embodiments are directed to methods wherein said Th17 cells express markers associated with metastasis.

Further embodiments are directed to methods wherein said metastasis associated marker is CD82.

Further embodiments are directed to methods wherein said Th17 cells express markers associated with inflammation.

Further embodiments are directed to methods wherein said marker associated with inflammation is IL-1 RAcP.

Further embodiments are directed to methods wherein said marker associated with inflammation is IL-18 receptor alpha.

Further embodiments are directed to methods wherein said marker associated with inflammation is TNF-alpha receptor p55.

Further embodiments are directed to methods wherein said marker associated with inflammation is CTLA-4.

Further embodiments are directed to methods wherein said marker associated with inflammation is BTLA-4.

Further embodiments are directed to methods wherein said marker associated with inflammation is IL-1 receptor antagonist.

Further embodiments are directed to methods wherein said marker associated with inflammation is CD144

Further embodiments are directed to methods wherein said Th17 cells express developmental associated markers.

Further embodiments are directed to methods wherein said development associated marker is Notch-1.

Further embodiments are directed to methods wherein said development associated marker is DLL3.

Further embodiments are directed to methods wherein said development associated marker is SSEA3.

Further embodiments are directed to methods wherein said development associated marker is SSEA4.

Further embodiments are directed to methods wherein said development associated marker is Nanog.

Further embodiments are directed to methods wherein said development associated marker is Sox-2.

Further embodiments are directed to methods wherein said development associated marker is Notch-1.

Further embodiments are directed to methods wherein said development associated marker is FGF-1 receptor.

Further embodiments are directed to methods wherein said development associated marker is frizzled.

Further embodiments are directed to methods wherein said development associated marker is FGF-2 receptor.

Further embodiments are directed to methods wherein said development associated marker is FGF-5.

Further embodiments are directed to methods wherein said development associated marker is PDGF-AA receptor.

Further embodiments are directed to methods wherein said development associated marker is PDGF-BB receptor.

Further embodiments are directed to methods wherein said development associated marker is EGF-1 receptor.

Further embodiments are directed to methods wherein said Th17 cells express markers associated with axon growth.

Further embodiments are directed to methods wherein said marker is Sema7A.

Further embodiments are directed to methods wherein said marker is Sema4D.

Further embodiments are directed to methods wherein said Th17 cells express lymphocyte markers.

Further embodiments are directed to methods wherein said lymphocyte marker is CD4.

Further embodiments are directed to methods wherein said lymphocyte marker is CD43.

Further embodiments are directed to methods wherein said lymphocyte marker is CD99.

Further embodiments are directed to methods wherein said lymphocyte marker is NTB -A.

Further embodiments are directed to methods wherein said lymphocyte marker is LAIR1.

Further embodiments are directed to methods wherein said lymphocyte marker is CCRL2.

Further embodiments are directed to methods wherein said Th17 cells express markers found on myeloid cells.

Further embodiments are directed to methods wherein said myeloid marker found on Th17 cells is CD200.

Further embodiments are directed to methods wherein said Th17 cells express markers found on thrombocytes.

Further embodiments are directed to methods wherein said markers found on thrombocytes is CD49f.

Further embodiments are directed to methods wherein said Th17 cells express markers found on erythrocytes.

Further embodiments are directed to methods wherein said marker found on erythrocytes is TRA-1-85.

Further embodiments are directed to methods wherein said Th17 cells express TfR.

Further embodiments are directed to methods wherein said Th17 cells express A33.

Further embodiments are directed to methods wherein said Th17 cells express adhesion and/or signaling molecules.

Further embodiments are directed to methods wherein said adhesion and/or signaling molecules found on Th17 cells are CD97.

Further embodiments are directed to methods wherein said adhesion and/or signaling molecules found on Th17 cells are CD53.

Further embodiments are directed to methods wherein said adhesion and/or signaling molecules found on Th17 cells are DEP-1.

Further embodiments are directed to methods wherein said adhesion and/or signaling molecules found on Th17 cells are ALCAM.

Further embodiments are directed to methods wherein said adhesion and/or signaling molecules found on Th17 cells are DNAM-1.

Further embodiments are directed to methods wherein said adhesion and/or signaling molecules found on Th17 cells are CD48.

Further embodiments are directed to methods wherein said adhesion and/or signaling molecules found on Th17 cells are IGF-II receptor

Further embodiments are directed to methods wherein said adhesion and/or signaling molecules found on Th17 cells are LRP-6.

The method of claim 1, wherein said marker associated with suicidal ideation/attempts is a reduction in T regulatory cells.

Further embodiments are directed to methods wherein said T regulatory cell is a cell expressing a transcription factor capable of inducing a suppressive phenotype upon T cells.

Further embodiments are directed to methods wherein said transcription factor is FoxP3.

Further embodiments are directed to methods wherein said T regulatory cell expresses CD4.

Further embodiments are directed to methods wherein said T regulatory cell expresses CD25.

Further embodiments are directed to methods wherein said T regulatory cell expresses Helios.

Further embodiments are directed to methods wherein said T regulatory cell expresses GITR ligand.

Further embodiments are directed to methods wherein said T regulatory cell expresses membrane bound TGF-beta.

Further embodiments are directed to methods wherein said T regulatory cell expresses Fas ligand.

Further embodiments are directed to methods wherein said T regulatory cell expresses CTLA-4.

Further embodiments are directed to methods wherein said T regulatory cell expresses IL-10.

Further embodiments are directed to methods wherein said T regulatory cell expresses IL-35.

Further embodiments are directed to methods wherein said T regulatory cell is capable of suppressing activation of a naïve T cell.

Further embodiments are directed to methods wherein said activation of said naïve cell is proliferation of said naïve cell.

Further embodiments are directed to methods wherein said activation of said naïve cell is production of cytokines from said naïve cell.

Further embodiments are directed to methods wherein said cytokines produced by said activation of said naïve cell are selected from a group comprising of: a) IL-2; b) IL-4; c) IL-7; d) IL-9; e) IL-10; f) IL-13; g) IL-15; h) IL-17; i) IL-18; j) IL-20; k) IL-23; l) IL-27; m) interferon gamma; and n) TNF-alpha.

Further embodiments are directed to methods wherein said activation of said naïve cell is acquisition of a memory cell phenotype.

Further embodiments are directed to methods wherein said activation of said naïve cell is acquisition of ability to induce differentiation of an immature dendritic cell.

Further embodiments are directed to methods wherein said differentiation of an immature dendritic cell is upregulation of CD40 expression.

Further embodiments are directed to methods wherein said differentiation of an immature dendritic cell is upregulation of CD80 expression.

Further embodiments are directed to methods wherein said differentiation of an immature dendritic cell is upregulation of CD86 expression.

Further embodiments are directed to methods wherein said differentiation of an immature dendritic cell is upregulation of neuropilin expression.

Further embodiments are directed to methods wherein said differentiation of an immature dendritic cell is upregulation of HLA-DR expression.

Further embodiments are directed to methods wherein said differentiation of an immature dendritic cell is upregulation of IL-12 expression.

Further embodiments are directed to methods wherein said differentiation of an immature dendritic cell is upregulation of lymph node homing chemokine receptor expression.

Further embodiments are directed to methods wherein said differentiation of an immature dendritic cell is downregulation of phagocytic activity.

Further embodiments are directed to methods wherein said differentiation of an immature dendritic cell is downregulation of IL-10 expression.

Further embodiments are directed to methods wherein said differentiation of an immature dendritic cell is upregulation of antigen presentation activity.

Further embodiments are directed to methods wherein said antigen presentation activity is ability to induce activation of a naïve T cell.

The method of claim 1 wherein said platelet rich plasma is significantly devoid of neutrophils.

The method of claim 1 wherein said platelet rich plasma possesses growth factors.

Further embodiments are directed to methods wherein said platelet rich plasma is filtered for enhancing concentration of specific growth factors.

Further embodiments are directed to methods wherein said concentration of said platelet rich plasma is performed by an affinity means.

Further embodiments are directed to methods wherein said affinity means is affinity chromatography.

Further embodiments are directed to methods wherein said affinity means is immunoadsorption.

Further embodiments are directed to methods wherein said affinity means is high pressure liquid chromatography.

Further embodiments are directed to methods wherein said affinity means is fast pressure liquid chromatography.

Further embodiments are directed to methods wherein growth factor concentrated from platelet rich plasma is Angiopoietin-like Protein 1/ANGPTL1.

Further embodiments are directed to methods wherein growth factor concentrated from platelet rich plasma is Angiopoietin-like Protein 2/ANGPTL2.

Further embodiments are directed to methods wherein growth factor concentrated from platelet rich plasma is Angiopoietin-like Protein 3/ANGPTL3.

Further embodiments are directed to methods wherein growth factor concentrated from platelet rich plasma is Angiopoietin-like Protein 4/ANGPTL4.

Further embodiments are directed to methods wherein growth factor concentrated from platelet rich plasma is Angiopoietin-like Protein 5/ANGPTL5.

Further embodiments are directed to methods wherein growth factor concentrated from platelet rich plasma is Angiopoietin-like Protein 6/ANGPTL6.

Further embodiments are directed to methods wherein growth factor concentrated from platelet rich plasma is Angiopoietin-like Protein 7/ANGPTL7.

Further embodiments are directed to methods wherein growth factor concentrated from platelet rich plasma is Angiopoietin-like Protein 8/Betatrophin.

Further embodiments are directed to methods wherein growth factor concentrated from platelet rich plasma is Angiopoietin-1.

Further embodiments are directed to methods wherein growth factor concentrated from platelet rich plasma is Angiopoietin-2.

Further embodiments are directed to methods wherein growth factor concentrated from platelet rich plasma is Angiopoietin-3.

Further embodiments are directed to methods wherein growth factor concentrated from platelet rich plasma is Angiopoietin-4.

Further embodiments are directed to methods wherein growth factor concentrated from platelet rich plasma is Amphiregulin.

Further embodiments are directed to methods wherein growth factor concentrated from platelet rich plasma is Betacellulin/BTC.

Further embodiments are directed to methods wherein growth factor concentrated from platelet rich plasma is EGF.

Further embodiments are directed to methods wherein growth factor concentrated from platelet rich plasma is EGF-L6.

Further embodiments are directed to methods wherein growth factor concentrated from platelet rich plasma is Epigen.

Further embodiments are directed to methods wherein growth factor concentrated from platelet rich plasma is Epiregulin.

Further embodiments are directed to methods wherein growth factor concentrated from platelet rich plasma is HB-EGF.

Further embodiments are directed to methods wherein growth factor concentrated from platelet rich plasma is LRIG1.

Further embodiments are directed to methods wherein growth factor concentrated from platelet rich plasma is LRIG2.

Further embodiments are directed to methods wherein growth factor concentrated from platelet rich plasma is LRIG3.

Further embodiments are directed to methods wherein growth factor concentrated from platelet rich plasma is Neuregulin-1/NRG1.

Further embodiments are directed to methods wherein growth factor concentrated from platelet rich plasma is Neuregulin-1 alpha/NRG1 alpha.

Further embodiments are directed to methods wherein growth factor concentrated from platelet rich plasma is Neuregulin-1 beta 1/NRG1 beta 1.

Further embodiments are directed to methods wherein growth factor concentrated from platelet rich plasma is Neuregulin-1 Isoform GGF2.

Further embodiments are directed to methods wherein growth factor concentrated from platelet rich plasma is Neuregulin-1 Isoform SMDF.

Further embodiments are directed to methods wherein growth factor concentrated from platelet rich plasma is Neuregulin-3/NRG3.

Further embodiments are directed to methods wherein growth factor concentrated from platelet rich plasma is TGF-alpha.

Further embodiments are directed to methods wherein growth factor concentrated from platelet rich plasma is TMEFF1/Tomoregulin-1.

Further embodiments are directed to methods wherein growth factor concentrated from platelet rich plasma is TMEFF2/Tomoregulin-2.

Further embodiments are directed to methods wherein growth factor concentrated from platelet rich plasma is EphA1.

Further embodiments are directed to methods wherein growth factor concentrated from platelet rich plasma is EphA2.

Further embodiments are directed to methods wherein growth factor concentrated from platelet rich plasma is EphA3.

Further embodiments are directed to methods wherein growth factor concentrated from platelet rich plasma is EphA4.

Further embodiments are directed to methods wherein growth factor concentrated from platelet rich plasma is EphA5.

Further embodiments are directed to methods wherein growth factor concentrated from platelet rich plasma is EphA6.

Further embodiments are directed to methods wherein growth factor concentrated from platelet rich plasma is EphA7.

Further embodiments are directed to methods wherein growth factor concentrated from platelet rich plasma is EphA8.

Further embodiments are directed to methods wherein growth factor concentrated from platelet rich plasma is EphA10.

Further embodiments are directed to methods wherein growth factor concentrated from platelet rich plasma is EphB.

Further embodiments are directed to methods wherein growth factor concentrated from platelet rich plasma is EphB1.

Further embodiments are directed to methods wherein growth factor concentrated from platelet rich plasma is EphB2.

Further embodiments are directed to methods wherein growth factor concentrated from platelet rich plasma is EphB3.

Further embodiments are directed to methods wherein growth factor concentrated from platelet rich plasma is EphB4.

Further embodiments are directed to methods wherein growth factor concentrated from platelet rich plasma is EphB6.

Further embodiments are directed to methods wherein growth factor concentrated from platelet rich plasma is Ephrin-A.

Further embodiments are directed to methods wherein growth factor concentrated from platelet rich plasma is Ephrin-A1.

Further embodiments are directed to methods wherein growth factor concentrated from platelet rich plasma is Ephrin-A2.

Further embodiments are directed to methods wherein growth factor concentrated from platelet rich plasma is Ephrin-A3.

Further embodiments are directed to methods wherein growth factor concentrated from platelet rich plasma is Ephrin-A4.

Further embodiments are directed to methods wherein growth factor concentrated from platelet rich plasma is Ephrin-A5.

Further embodiments are directed to methods wherein growth factor concentrated from platelet rich plasma is Ephrin-B.

Further embodiments are directed to methods wherein growth factor concentrated from platelet rich plasma is Ephrin-B.

Further embodiments are directed to methods wherein growth factor concentrated from platelet rich plasma is Ephrin-B1

Further embodiments are directed to methods wherein growth factor concentrated from platelet rich plasma is Ephrin-B2.

Further embodiments are directed to methods wherein growth factor concentrated from platelet rich plasma is Ephrin-B3.

Further embodiments are directed to methods wherein growth factor concentrated from platelet rich plasma is FGF-1.

Further embodiments are directed to methods wherein growth factor concentrated from platelet rich plasma is FGF-2.

Further embodiments are directed to methods wherein growth factor concentrated from platelet rich plasma is FGF-3.

Further embodiments are directed to methods wherein growth factor concentrated from platelet rich plasma is FGF-4.

Further embodiments are directed to methods wherein growth factor concentrated from platelet rich plasma is FGF-5.

Further embodiments are directed to methods wherein growth factor concentrated from platelet rich plasma is FGF-6

Further embodiments are directed to methods wherein growth factor concentrated from platelet rich plasma is KGF.

Further embodiments are directed to methods wherein growth factor concentrated from platelet rich plasma is FGF-8.

Further embodiments are directed to methods wherein growth factor concentrated from platelet rich plasma is FGF-9.

Further embodiments are directed to methods wherein growth factor concentrated from platelet rich plasma is FGF-10.

Further embodiments are directed to methods wherein growth factor concentrated from platelet rich plasma is FGF-11.

Further embodiments are directed to methods wherein growth factor concentrated from platelet rich plasma is FGF-12.

Further embodiments are directed to methods wherein growth factor concentrated from platelet rich plasma is FGF-13.

Further embodiments are directed to methods wherein growth factor concentrated from platelet rich plasma is FGF-15.

Further embodiments are directed to methods wherein growth factor concentrated from platelet rich plasma is FGF-16.

Further embodiments are directed to methods wherein growth factor concentrated from platelet rich plasma is FGF-17.

Further embodiments are directed to methods wherein growth factor concentrated from platelet rich plasma is FGF-18.

Further embodiments are directed to methods wherein growth factor concentrated from platelet rich plasma is FGF-19.

Further embodiments are directed to methods wherein growth factor concentrated from platelet rich plasma is FGF-20.

Further embodiments are directed to methods wherein growth factor concentrated from platelet rich plasma is FGF-21.

Further embodiments are directed to methods wherein growth factor concentrated from platelet rich plasma is FGF-22.

Further embodiments are directed to methods wherein growth factor concentrated from platelet rich plasma is FGF-23.

Further embodiments are directed to methods wherein growth factor concentrated from platelet rich plasma is alpha 2-Macroglobulin.

Further embodiments are directed to methods wherein growth factor concentrated from platelet rich plasma is alpha 2-Macroglobulin-like 1/A2ML1.

Further embodiments are directed to methods wherein growth factor concentrated from platelet rich plasma is CNPY2.

Further embodiments are directed to methods wherein growth factor concentrated from platelet rich plasma is FGF-BP.

Further embodiments are directed to methods wherein growth factor concentrated from platelet rich plasma is FGFBP2.

Further embodiments are directed to methods wherein growth factor concentrated from platelet rich plasma is FGFBP3.

Further embodiments are directed to methods wherein growth factor concentrated from platelet rich plasma is FRS2.

Further embodiments are directed to methods wherein growth factor concentrated from platelet rich plasma is Klotho.

Further embodiments are directed to methods wherein growth factor concentrated from platelet rich plasma is Klotho beta.

Further embodiments are directed to methods wherein growth factor concentrated from platelet rich plasma is LRIT3.

Further embodiments are directed to methods wherein growth factor concentrated from platelet rich plasma is Pentraxin 3/TSG-14.

Further embodiments are directed to methods wherein growth factor concentrated from platelet rich plasma is Shisa-4.

Further embodiments are directed to methods wherein growth factor concentrated from platelet rich plasma is SPRY1.

Further embodiments are directed to methods wherein growth factor concentrated from platelet rich plasma is SPRY2.

Further embodiments are directed to methods wherein growth factor concentrated from platelet rich plasma is SPRY3.

Further embodiments are directed to methods wherein growth factor concentrated from platelet rich plasma is Desert Hedgehog/Dhh.

Further embodiments are directed to methods wherein growth factor concentrated from platelet rich plasma is Indian Hedgehog/Ihh.

Further embodiments are directed to methods wherein growth factor concentrated from platelet rich plasma is Sonic Hedgehog/Shh.

Further embodiments are directed to methods wherein growth factor concentrated from platelet rich plasma is Hedgehog Related Molecules and Regulators.

Further embodiments are directed to methods wherein growth factor concentrated from platelet rich plasma is beta-TrCP1/BTRC.

Further embodiments are directed to methods wherein growth factor concentrated from platelet rich plasma is BOC.

Further embodiments are directed to methods wherein growth factor concentrated from platelet rich plasma is C2CD3.

Further embodiments are directed to methods wherein growth factor concentrated from platelet rich plasma is CDO.

Further embodiments are directed to methods wherein growth factor concentrated from platelet rich plasma is DISP1.

Further embodiments are directed to methods wherein growth factor concentrated from platelet rich plasma is DISP2.

Further embodiments are directed to methods wherein growth factor concentrated from platelet rich plasma is Gas1.

Further embodiments are directed to methods wherein growth factor concentrated from platelet rich plasma is GLI-1.

Further embodiments are directed to methods wherein growth factor concentrated from platelet rich plasma is GLI-2.

Further embodiments are directed to methods wherein growth factor concentrated from platelet rich plasma is GLI-3.

Further embodiments are directed to methods wherein growth factor concentrated from platelet rich plasma is Glypican 3.

Further embodiments are directed to methods wherein growth factor concentrated from platelet rich plasma is Glypican 4.

Further embodiments are directed to methods wherein growth factor concentrated from platelet rich plasma is GPR161.

Further embodiments are directed to methods wherein growth factor concentrated from platelet rich plasma is GSK-3 alpha/beta.

Further embodiments are directed to methods wherein growth factor concentrated from platelet rich plasma is GSK-3 alpha.

Further embodiments are directed to methods wherein growth factor concentrated from platelet rich plasma is GSK-3 beta.

Further embodiments are directed to methods wherein growth factor concentrated from platelet rich plasma is Hedgehog Signaling Activators.

Further embodiments are directed to methods wherein growth factor concentrated from platelet rich plasma is Hedgehog Signaling Inhibitors.

Further embodiments are directed to methods wherein growth factor concentrated from platelet rich plasma is HHAT.

Further embodiments are directed to methods wherein growth factor concentrated from platelet rich plasma is HHIPL1.

Further embodiments are directed to methods wherein growth factor concentrated from platelet rich plasma is HHIPL2.

Further embodiments are directed to methods wherein growth factor concentrated from platelet rich plasma is Hip.

Further embodiments are directed to methods wherein growth factor concentrated from platelet rich plasma is LIN-41.

Further embodiments are directed to methods wherein growth factor concentrated from platelet rich plasma is Patched 1/PTCH.

Further embodiments are directed to methods wherein growth factor concentrated from platelet rich plasma is Patched 2/PTCH2.

Further embodiments are directed to methods wherein growth factor concentrated from platelet rich plasma is SCUBE3.

Further embodiments are directed to methods wherein growth factor concentrated from platelet rich plasma is smoothened rp1/IGFBP-7.

Further embodiments are directed to methods wherein growth factor concentrated from platelet rich plasma is TCTN1.

Further embodiments are directed to methods wherein growth factor concentrated from platelet rich plasma is HTRA4.

Further embodiments are directed to methods wherein growth factor concentrated from platelet rich plasma is IGFBP-1.

Further embodiments are directed to methods wherein growth factor concentrated from platelet rich plasma is IGFBP-2.

Further embodiments are directed to methods wherein growth factor concentrated from platelet rich plasma is IGFBP-3.

Further embodiments are directed to methods wherein growth factor concentrated from platelet rich plasma is IGFBP-4.

Further embodiments are directed to methods wherein growth factor concentrated from platelet rich plasma is IGFBP-5.

Further embodiments are directed to methods wherein growth factor concentrated from platelet rich plasma is IGFBP-6.

Further embodiments are directed to methods wherein growth factor concentrated from platelet rich plasma is IGFBP-

Further embodiments are directed to methods wherein growth factor concentrated from platelet rich plasma is IGFBP-

Further embodiments are directed to methods wherein growth factor concentrated from platelet rich plasma is IGFBP-rP10

Further embodiments are directed to methods wherein growth factor concentrated from platelet rich plasma is IGF-I/IGF-1.

Further embodiments are directed to methods wherein growth factor concentrated from platelet rich plasma is IGF-II/IGF2

Further embodiments are directed to methods wherein growth factor concentrated from platelet rich plasma is IGFL-3

Further embodiments are directed to methods wherein growth factor concentrated from platelet rich plasma is CILP-1

Further embodiments are directed to methods wherein growth factor concentrated from platelet rich plasma is CTGF/CCN2.

Further embodiments are directed to methods wherein growth factor concentrated from platelet rich plasma is Cyr61/CCN1

Further embodiments are directed to methods wherein growth factor concentrated from platelet rich plasma is Endocan/ESM-1.

Further embodiments are directed to methods wherein growth factor concentrated from platelet rich plasma is IGFALS/ALS.

Further embodiments are directed to methods wherein growth factor concentrated from platelet rich plasma is IMP2.

Further embodiments are directed to methods wherein growth factor concentrated from platelet rich plasma is NOV/CCN3.

Further embodiments are directed to methods wherein growth factor concentrated from platelet rich plasma is TMEM219.

Further embodiments are directed to methods wherein growth factor concentrated from platelet rich plasma is WISP-1/CCN4.

Further embodiments are directed to methods wherein growth factor concentrated from platelet rich plasma is WISP3

Further embodiments are directed to methods wherein growth factor concentrated from platelet rich plasma is PDGF-A.

Further embodiments are directed to methods wherein growth factor concentrated from platelet rich plasma is PDGF-AA.

Further embodiments are directed to methods wherein growth factor concentrated from platelet rich plasma is PDGF-AB.

Further embodiments are directed to methods wherein growth factor concentrated from platelet rich plasma is PDGF-AB/BB.

Further embodiments are directed to methods wherein growth factor concentrated from platelet rich plasma is PDGF-B.

Further embodiments are directed to methods wherein growth factor concentrated from platelet rich plasma is PDGF-BB.

Further embodiments are directed to methods wherein growth factor concentrated from platelet rich plasma is PDGF-C.

Further embodiments are directed to methods wherein growth factor concentrated from platelet rich plasma is PDGF-CC.

Further embodiments are directed to methods wherein growth factor concentrated from platelet rich plasma is PDGF-D.

Further embodiments are directed to methods wherein growth factor concentrated from platelet rich plasma is PDGF-DD.

Further embodiments are directed to methods wherein growth factor concentrated from platelet rich plasma is HGF-1. 601. Further embodiments are directed to methods wherein growth factor concentrated from platelet rich plasma is Decorin.

Further embodiments are directed to methods wherein growth factor concentrated from platelet rich plasma is Glypican 1.

Further embodiments are directed to methods wherein growth factor concentrated from platelet rich plasma is Glypican 2.

Further embodiments are directed to methods wherein growth factor concentrated from platelet rich plasma is Glypican 3.

Further embodiments are directed to methods wherein growth factor concentrated from platelet rich plasma is Glypican 4.

Further embodiments are directed to methods wherein growth factor concentrated from platelet rich plasma is Glypican 5.

Further embodiments are directed to methods wherein growth factor concentrated from platelet rich plasma is Glypican 6.

Further embodiments are directed to methods wherein growth factor concentrated from platelet rich plasma is aggrecan.

Further embodiments are directed to methods wherein growth factor concentrated from platelet rich plasma is Brevican.

Further embodiments are directed to methods wherein growth factor concentrated from platelet rich plasma is Neurocan.

Further embodiments are directed to methods wherein growth factor concentrated from platelet rich plasma is versican.

Further embodiments are directed to methods wherein growth factor concentrated from platelet rich plasma is Syndecan-1/CD138.

Further embodiments are directed to methods wherein growth factor concentrated from platelet rich plasma is Syndecan-2/CD362.

Further embodiments are directed to methods wherein growth factor concentrated from platelet rich plasma is Syndecan-3.

Further embodiments are directed to methods wherein growth factor concentrated from platelet rich plasma is Syndecan-4

Further embodiments are directed to methods wherein growth factor concentrated from platelet rich plasma is Testican 1/SPOCK1.

Further embodiments are directed to methods wherein growth factor concentrated from platelet rich plasma is Testican 2/SPOCK2.

Further embodiments are directed to methods wherein growth factor concentrated from platelet rich plasma is Testican 3/SPOCK3.

Further embodiments are directed to methods wherein growth factor concentrated from platelet rich plasma is Agrin.

Further embodiments are directed to methods wherein growth factor concentrated from platelet rich plasma is alpha-Sarcoglycan.

Further embodiments are directed to methods wherein growth factor concentrated from platelet rich plasma is beta-Sarcoglycan.

Further embodiments are directed to methods wherein growth factor concentrated from platelet rich plasma is Biglycan.

Further embodiments are directed to methods wherein growth factor concentrated from platelet rich plasma is Bikunin.

Further embodiments are directed to methods wherein growth factor concentrated from platelet rich plasma is CHADL.

Further embodiments are directed to methods wherein growth factor concentrated from platelet rich plasma is Chondroadherin.

Further embodiments are directed to methods wherein growth factor concentrated from platelet rich plasma is Cytokeratin 18.

Further embodiments are directed to methods wherein growth factor concentrated from platelet rich plasma is DSPG3.

Further embodiments are directed to methods wherein growth factor concentrated from platelet rich plasma is Dystroglycan.

Further embodiments are directed to methods wherein growth factor concentrated from platelet rich plasma is Endocan/ESM-1.

Further embodiments are directed to methods wherein growth factor concentrated from platelet rich plasma is Endoglycan/PODXL2.

Further embodiments are directed to methods wherein growth factor concentrated from platelet rich plasma is Endorepellin/Perlecan.

Further embodiments are directed to methods wherein growth factor concentrated from platelet rich plasma is epsilon-Sarcoglycan.

Further embodiments are directed to methods wherein growth factor concentrated from platelet rich plasma is Fibromodulin/FMOD.

Further embodiments are directed to methods wherein growth factor concentrated from platelet rich plasma is Keratocan.

Further embodiments are directed to methods wherein growth factor concentrated from platelet rich plasma is Lumican.

Further embodiments are directed to methods wherein growth factor concentrated from platelet rich plasma is MBP-1.

Further embodiments are directed to methods wherein growth factor concentrated from platelet rich plasma is Mimecan.

Further embodiments are directed to methods wherein growth factor concentrated from platelet rich plasma is Neuroglycan C/CSPG5.

Further embodiments are directed to methods wherein growth factor concentrated from platelet rich plasma is NG2/MCSP.

Further embodiments are directed to methods wherein growth factor concentrated from platelet rich plasma is NYX.

Further embodiments are directed to methods wherein growth factor concentrated from platelet rich plasma is Opticin.

Further embodiments are directed to methods wherein growth factor concentrated from platelet rich plasma is Osteoadherin/OSAD.

Further embodiments are directed to methods wherein growth factor concentrated from platelet rich plasma is Podocan.

Further embodiments are directed to methods wherein growth factor concentrated from platelet rich plasma is PRELP.

Further embodiments are directed to methods wherein growth factor concentrated from platelet rich plasma is PRG3/MBP2.

Further embodiments are directed to methods wherein growth factor concentrated from platelet rich plasma is delta-Sarcoglycan.

Further embodiments are directed to methods wherein growth factor concentrated from platelet rich plasma is TGF-beta RIII.

Further embodiments are directed to methods wherein growth factor concentrated from platelet rich plasma is PlGF-2.

Further embodiments are directed to methods wherein growth factor concentrated from platelet rich plasma is PlGF-3.

Further embodiments are directed to methods wherein growth factor concentrated from platelet rich plasma is PlGF-4.

Further embodiments are directed to methods wherein growth factor concentrated from platelet rich plasma is VEGF/PlGF Heterodimer.

Further embodiments are directed to methods wherein growth factor concentrated from platelet rich plasma is VEGF-B.

Further embodiments are directed to methods wherein growth factor concentrated from platelet rich plasma is VEGF-C.

Further embodiments are directed to methods wherein growth factor concentrated from platelet rich plasma is VEGF-D

Further embodiments are directed to methods wherein growth factor concentrated from platelet rich plasma is Chondromodulin-1.

Further embodiments are directed to methods wherein growth factor concentrated from platelet rich plasma is CTGF/CCN2.

Further embodiments are directed to methods wherein growth factor concentrated from platelet rich plasma is Cyr61/CCN1.

Further embodiments are directed to methods wherein growth factor concentrated from platelet rich plasma is EG-VEGF/PK1.

Further embodiments are directed to methods wherein growth factor concentrated from platelet rich plasma is Fibulin 1.

Further embodiments are directed to methods wherein growth factor concentrated from platelet rich plasma is Fibulin 2.

Further embodiments are directed to methods wherein growth factor concentrated from platelet rich plasma is Fibulin 3.

Further embodiments are directed to methods wherein growth factor concentrated from platelet rich plasma is Fibulin 5/DANCE.

Further embodiments are directed to methods wherein growth factor concentrated from platelet rich plasma is Fibulin 7.

Further embodiments are directed to methods wherein growth factor concentrated from platelet rich plasma is HDGF

Further embodiments are directed to methods wherein growth factor concentrated from platelet rich plasma is Hepassocin/FGL1.

Further embodiments are directed to methods wherein growth factor concentrated from platelet rich plasma is LECT2. Further embodiments are directed to methods wherein growth factor concentrated from platelet rich plasma is LEDGF.

Further embodiments are directed to methods wherein growth factor concentrated from platelet rich plasma is LRRN1/NLRR-1.

Further embodiments are directed to methods wherein growth factor concentrated from platelet rich plasma is LYAR.

Further embodiments are directed to methods wherein growth factor concentrated from platelet rich plasma is beta-NGF.

Further embodiments are directed to methods wherein growth factor concentrated from platelet rich plasma is Norrin.

Further embodiments are directed to methods wherein growth factor concentrated from platelet rich plasma is NOV/CCN3.

Further embodiments are directed to methods wherein growth factor concentrated from platelet rich plasma is Oncomodulin

Further embodiments are directed to methods wherein growth factor concentrated from platelet rich plasma is Osteocrin.

Further embodiments are directed to methods wherein growth factor concentrated from platelet rich plasma is PD-ECGF.

Further embodiments are directed to methods wherein growth factor concentrated from platelet rich plasma is Progranulin/PGRN.

Further embodiments are directed to methods wherein growth factor concentrated from platelet rich plasma is S100A13.

Further embodiments are directed to methods wherein growth factor concentrated from platelet rich plasma is SF20/MYDGF.

Further embodiments are directed to methods wherein growth factor concentrated from platelet rich plasma is SLURP1.

Further embodiments are directed to methods wherein growth factor concentrated from platelet rich plasma is SLURP2.

Further embodiments are directed to methods wherein growth factor concentrated from platelet rich plasma is Thrombopoietin/Tpo.

Further embodiments are directed to methods wherein growth factor concentrated from platelet rich plasma is WISP-1/CCN4

Further embodiments are directed to methods wherein growth factor concentrated from platelet rich plasma is WISP3.

Further embodiments are directed to methods wherein said anti-inflammatory/regenerative adjuvant is Calagualine (fern derivative).

Further embodiments are directed to methods wherein said anti-inflammatory/regenerative adjuvant is Conophylline (Ervatamia microphylla).

Further embodiments are directed to methods wherein said anti-inflammatory/regenerative adjuvant is Evodiamine (Evodiae fructus component).

Further embodiments are directed to methods wherein said anti-inflammatory/regenerative adjuvant is Geldanamycin.

Further embodiments are directed to methods wherein said anti-inflammatory/regenerative adjuvant is Perrilyl alcohol.

Further embodiments are directed to methods wherein said anti-inflammatory/regenerative adjuvant is pterostilbene.

Further embodiments are directed to methods wherein said anti-inflammatory/regenerative adjuvant is Protein-bound polysaccharide from basidiomycetes.

Further embodiments are directed to methods wherein said anti-inflammatory/regenerative adjuvant is Rocaglamides (Aglaia derivatives).

Further embodiments are directed to methods wherein said anti-inflammatory/regenerative adjuvant is 15-deoxy-prostaglandin J(2).

Further embodiments are directed to methods wherein said anti-inflammatory/regenerative adjuvant is lithium.

Further embodiments are directed to methods wherein said anti-inflammatory/regenerative adjuvant is Anandamide. Further embodiments are directed to methods wherein said anti-inflammatory/regenerative adjuvant is Artemisia vestita. Further embodiments are directed to methods wherein said anti-inflammatory/regenerative adjuvant is Cobrotoxin.

Further embodiments are directed to methods wherein said anti-inflammatory/regenerative adjuvant is Dehydroascorbic acid (Vitamin C).

Further embodiments are directed to methods wherein said anti-inflammatory/regenerative adjuvant is Herbimycin A.

Further embodiments are directed to methods wherein said anti-inflammatory/regenerative adjuvant is Isorhapontigenin.

Further embodiments are directed to methods wherein said anti-inflammatory/regenerative adjuvant is Manumycin A.

Further embodiments are directed to methods wherein said anti-inflammatory/regenerative adjuvant is Pomegranate fruit extract.

Further embodiments are directed to methods wherein said anti-inflammatory/regenerative adjuvant is selected from a group comprising of: Tetrandine (plant alkaloid), Thienopyridine, Acetyl-boswellic acids, 1′- Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

1-711. (canceled)

712. A method of reducing suicidal ideations/suicidal attempts comprising the steps of:
a) obtaining a patient with a propensity for suicidal ideation/suicidal attempts; b) assessing one or more markers associated with said suicidal ideations/suicidal attempts; c) administering to said patient platelet rich plasma and/or cord blood plasma alone or in combination with an anti-inflammatory/regenerative adjuvant; d) assessing levels of said one or more markers associated with said suicidal ideations/suicidal attempts; and e) adjusting dosage/frequency of administration of said platelet rich plasma alone or in combination with an antiinflammatory/regenerative adjuvant.

713. The method of claim 712, wherein propensity for suicidal ideation/suicidal attempts is associated with augmentation of inflammatory cytokines and/or mediators in a biological fluid as compared to an age matched control.

714. The method of claim 713, wherein said inflammatory cytokines are selected from a group consisting of: a) IL-1 beta; b) IL-6; c) TNF-alpha; d) IL-18; e) IL-17; f) IL-33 and g) interferon gamma.

715. The method of claim 712, wherein said anti-inflammatory/regenerative adjuvant is human chorionic gonadotropin.

716. The method of claim 712, wherein said anti-inflammatory/regenerative adjuvant is oxytocin.

717. The method of claim 712, wherein said anti-inflammatory/regenerative adjuvant is pterostilbene.

718. The method of claim 712, wherein said anti-inflammatory/regenerative adjuvant is G-CSF.

719. The method of claim 712, wherein said anti-inflammatory/regenerative adjuvant is GDF-11.

720. The method of claim 712, wherein said anti-inflammatory/regenerative adjuvant is pterostilbene delivered by nanoparticles.

721. The method of claim 712, wherein said anti-inflammatory/regenerative adjuvant is regenerative cell conditioned media.

722. The method of claim 721, wherein said conditioned media is concentrated for exosomes.

723. The method of claim 712, wherein said exosomes express CD9.

724. The method of claim 712, wherein said exosomes express soluble TNF receptor.

725. The method of claim 712, wherein said soluble TNF receptor is p55.

726. The method of claim 712, wherein said soluble TNF receptor is p75.

727. The method of claim 712, regenerative cells are hematopoietic stem cells.

728. The method of claim 712, regenerative cells are mesenchymal stem cells.

729. The method of claim 712, regenerative cells are dendritic cells.

730. The method of claim 712, regenerative cells are umbilical cord derived mesenchymal cells.

731. A method of treating suicidal ideations/suicide attempts by administration of platelet rich plasma together with pterostilbene.
